(19) United States
(12) Patent Application Publication
(10) Pub. No.: US 2008/0305966 A1
(43) Pub. Date: Dec. 11, 2008

(54) Capture Probe Design for Efficient Hybridisation

(76) Inventors: Regis Peytavi, Cabestany (FR); Frederic Raymond, Quebec (CA)

Correspondence Address: GODFREY & KAHN S.C., 780 NORTH WATER STREET, MILWAUKEE, WI, 53202, US

(21) Appl. No.: 11/573,183

(22) PCT Filed: Jun. 30, 2005
(86) PCT No.: PCT/CA2005/001030

§ 371 Date: Nov. 8, 2007

Related U.S. Application Data
(60) Provisional application No. 60/592,392, filed on Aug. 2, 2004.

(51) Int. Cl. C40B 40/06; C07H 21/04; G01N 33/50

(52) U.S. Cl. 506/16; 536/24.3; 436/94

(57) ABSTRACT

Methods for selecting and designing optimal nucleic acid-based probe for improving the sensitivity of detection of a nucleic acid-based target are disclosed herein. The capture probes generated from these methods show a significant improvement in the sensitivity of detection. Improved probes as well as microarrays and kits comprising these probes are disclosed herewith.

### FIELD OF THE INVENTION

The present invention relates to methods for selecting and designing optimal probe for improving the sensitivity of detection of a target as well as methods of detection. The present invention also provides capture probes, microarrays, kits comprising such probes.

### BACKGROUND OF THE INVENTION

Over the last decade, DNA microarrays have become useful tools in genomic studies and drug discovery (Debouck et al., 1999, Nat. Genet., 21:48-50; Duggan et al., 1999, Nat. Genet., 21:10-14; Marton et al., 1998, Nat. Med. 4:1293-1301). Unlike other hybridisation formats, microarrays allow significant miniaturisation, as thousands of different DNA fragments or oligonucleotide probes may be spotted onto a solid support, generally a glass slide. Other kinds of solid supports like plastic surfaces and porous microspheres may also be used. Therefore, microarrays are ideal for extensive gene profiling studies and multiplexed detection of nucleic acids for diagnostic purposes. While microarrays have been widely used in gene expression profiling, they also offer a great potential for the detection and identification of single nucleotide polymorphisms (SNPs) and for the diagnosis of infectious and genetic diseases. Examples of useful applications include cancer prognostics (Cardoso, 2003, Breast Cancer Res., 5:303-304; Cromer et al., 2004, Oncogene, 23:2484-2498), applications in forensic science (Verpoorte, 2002, Electrophoresis, 23:677-712), detection of microbes and their associated antimicrobial resistance genotypes (Mikhailovich et al., 2001, J. Clin. Microbiol., 39:2531-2540; Davies et al., 2002, FEMS Microbiol. Lett., 217:219-224), and detection of bio-weapon pathogens (Stenger et al., 2002, Curr. Opin. Biotechnol., 13:208-212).

While DNA probes longer than 70 nucleotides give reproducible hybridisation signals (Kane et al., 2000, Nucleic Acids Res., 38:4552-4557; Wang et al., 2002, FEMS Microbiol. Lett., 213:175-182), only short oligonucleotides (15-20 bases long) allow efficient discrimination of SNPs (Urakawa et al., 2003, Appl. Environ. Microbiol., 69:2848-2856; Guo et al., 1994, Nucleic Acids Res., 22:5456-5465). However, the hybridisation efficiency of shorter probes (i.e. less than 70 nucleotides) is still unpredictable, and false-negative results are often observed when short surface-bound DNA probes are used on microarrays. Many parameters are suspected to influence the hybridisation efficiency of target DNA to immobilised oligonucleotide DNA probes. These parameters include steric hindrance, secondary structure of the target DNA, and binding capacity of the surface-bound probe.

Steric hindrance may vary with probe density and spacer length, as well as with hydrophobicity and charge of the solid support (Chizhikov et al., 2001, Appl. Environ. Microbiol., 67:3258-3263). The secondary structure of the target DNA was shown to influence the efficiency of hybridisation and may be relieved by using helper oligonucleotides hybridising beside the probe (Wang et al., 2002, FEMS Microbiol. Lett., 213:175-182). The influence of the target secondary structure may be partially circumvented by selecting probes for their signal intensity and reproducibility (Peplies et al., 2003, Appl. Environ. Microbiol., 69:1397-1407) or for their theoretical thermodynamic behaviour (Matveeva et al., 2003, Nucleic Acids Res., 31:4211-4217). In addition, the use of single-stranded nucleic acid targets, instead of denatured, double-stranded amplicons, has been found to increase the sensitivity of hybridisation reactions using short capture probes suggesting that the complementary strand may interfere with the hybridisation of nucleic acid targets to the capture probes (Peplies et al., 2003, Appl. Environ. Microbiol., 69:1397-1407; Tao et al., 2003, Mol. Cell. Probes, 17:197-202; Gao et al., 2003, Analytical Letters, 33:2849-2863; Nikiforov et al., 1994, PCR Methods Appl., 3:285-291). Moreover, the design of oligonucleotide probes that are both sensitive and specific enough to discriminate SNPs is not easily predictable by the capture probe Tm (Wang et al., 2002, FEMS Microbiol. Lett., 213:175-182; Reyes-Lopez et al., 2003, Nucleic Acids Res., 31:779-789). Thus, oligonucleotide design is done either empirically (Southern et al., 1994, Nucleic Acids Res., 22:1368-1373; Antipova et al., 2002, Genome Biol., 3:research0073.1-research0073.4) or by using software based on heuristic algorithms (Lockhart et al., 1996, Nat. Biotechnol., 14:1675-1680).

There thus remains a need to improve the selection and design of optimal oligonucleotide capture probes for microarray hybridisation.

The present invention seeks to meet these and other needs.

The present description refers to a number of documents, the content of which is herein incorporated by reference in its entirety.

### SUMMARY OF THE INVENTION

The present invention provides methods for selecting and designing optimal nucleic acid-based probe for improving the sensitivity of detection of a nucleic acid-based target.

The present invention also provides capture probes allowing improvement in the sensitivity of detection of a target.

The present invention further provides detection methods based on the capture probes disclosed herein as well as microarrays and kits comprising such material.

In one aspect thereof, the present invention relates to a method of detecting at least one nucleic acid-based target, the method may comprise, contacting the target with a solid support-anchored oligonucleotide-based capture probe which may be able to bind a region located between nucleotide no. 1 and nucleotide no. n or between nucleotide no. m and nucleotide no. q of the target,
wherein n may be defined according to (calculated by) the formula n=0.4q (i.e., (n/q)×100=40%),
wherein m may be defined according to (calculated by) the formula m=0.6q (i.e., (m/q)×100=60%),
wherein q represents the total nucleotide number of the target (i.e., q corresponds to the last nucleotide of the target).

Upon hybridisation of the capture probe and target described herein, the unhybridised portion of the target which extends away (overhang) from the solid support to which it is linked may be about 40% or less of the total length (e.g., in nucleotides) of the target.

In accordance with the present invention, when the capture probe binds to a region located between nucleotide no. 1 and nucleotide no. n of the target the capture probe may be linked to the support by its 5′ end. Further in accordance with the present invention, when the capture probe binds to a region located between nucleotide no. m and nucleotide no. q of the target, the capture probe may be linked to the support by its 3′ end.

The target may be captured therefore by a 5′ anchored capture probe which may bind a region that lies closer to the 5′ end of the target. This capture probe may bind, for example, a region located within 40 percent of the length of the entire captured strand on its 5′ side.

For example, detection methods which use a capture probe which targets a region on a target nucleic acid strand so that, upon hybridisation of the probe and target, the longest part of the target strand may be oriented toward (is proximal to) the solid support to which the probe may be bound is encompassed herewith. In such methods, about at least 60% of a target's length may be proximal to the solid support to which the probe is bound. Therefore about, at least 40% of the target's length may be extending away from the support. The length of the probe is not intended herein to substantially influence any of the percentages discussed herein. For example, the probe may overlap the desired region of the target described herein as well as a region outside of the desired region.

Unless it is specifically mentioned otherwise, it is to be understood herein that the nucleotide numbering is attributed based on the 5′ to 3′ nomenclature. For example, nucleotide no. 1 represents the first nucleotide encountered starting from the 5′ end of a target, whether the target is the sense strand or the anti-sense strand of a double-stranded nucleic acid. Similarly, nucleotide numbering of n, m and q are attributed based on the 5′ to 3′ nomenclature.

It is also being understood herein that n, m and q are either integers or fractions which have been rounded to the closest integer. When n and/or m are for example 0.5, 1.5, etc., n and/or m are attributed the next upper integer, e.g., 1, 2, etc.

Using the method and probes of the present invention has been found to advantageously generate a higher detection signal in comparison to a signal measured for a second capture probe which binds to a region outside of the region located between nucleotide no. 1 and nucleotide no. n or between nucleotide no. m and nucleotide no. q of the target. For example, the signal intensity measured for the capture probe of the present invention is generally higher than the signal intensity which is measured for a second probe located outside of the desired region. Generally, the closer the region of the target to which the probe binds is to nucleotide no. 1 or nucleotide no. q of the target, the higher is a signal obtained with the method.

The method of the present invention may also comprise a step of detecting a complex formed by an hybridised capture probe and a target.

In accordance with the present invention, the target may comprise a detectable label (marker) such as a fluorescent label which generates, for example, a fluorescence signal which may be measured and/or quantified using methods, reagents and equipments known in the art.

Also in accordance with the present invention, the target may be from between 50 and 1000 nucleotides long. If desired the target may be longer than 1000 nucleotides. The proper location of the probe is also determined according to the method of the present invention.

In accordance with the present invention, the unhybridised portion (overhang) of the target may be, for example, less than 1000 nucleotides (i.e., for target longer than 1000 nucleotides, e.g., 2500 nucleotides). The unhybridised portion may be, for example, less than 750 nucleotides (e.g., less than 500 nucleotides, less than 300 nucleotides, less than 250 nucleotides, less than 200 nucleotides, less than 100 nucleotides, less than 50 nucleotides and even 0 (i.e., no overhang)). However, depending on the total length of the target, the method may even be applied to targets having an unhybridised portion of more than 1000 nucleotides.

In accordance with the present invention, the target may be a single-stranded nucleic acid. Further in accordance with the present invention, the target may be a denatured double-stranded nucleic acid. The target may be, for example, a PCR amplicon, genomic DNA, cDNA, RNA, etc. The target nucleic acid may be, for example, amplified DNA or reverse transcribed and PCR-amplified RNA. The target nucleic acids may be amplified by techniques (nucleic acid amplification technology) known in the art, such as, for example, PCR, RT-PCR (reverse transcription polymerase chain reaction), ligase chain reaction (LCR), transcription-mediated amplification (TMA), strand displacement amplification (SDA), etc.

In accordance with the present invention, the target product (e.g., PCR amplicon, DNA fragment, etc.) may be from about 50 to about 1000 nucleotides long (bases (nucleotides) or base pairs (bp)). The complex formed by the target and the probe may be detected (e.g., upon hydridisation) by methods known in the art. A detectale label (a fluorescent label, a fluorophore, etc.) may allow detection of the target. For example, a target DNA may be labelled with a fluorophore during PCR amplification (see Example 1). In addition, detection may be done, for example, using fluorescence, colorimetry, a physical process such as; plasmon resonance surface, microbalance, cantilever, mass spectrometry, electrochemistry, polymeric biosensors or any other detection methods. The signal may be detected and quantified using equipment known in the art including those described herein.

The method of the present invention may be more particularly applied to targets having an unhybridised portion which may be susceptible of being in contact with a substantially complementary sequence.

In accordance with the present invention, the method may be applied to hydridisation techniques which may need to be carried out about 15 minutes or more (e.g., more than 30 minutes).

Further in accordance with the present invention, the method may be used for capture probe having, for example, a ΔG of between 0 and −10 kcal/mol.

The method of the present invention may also be applied for the detection of at least two different types of target which are able to be captured by the probe. In such instance, the signal obtained for a first complex formed by a capture probe hybridised with a first type of target may be compared with the signal obtained for a second complex formed by the capture probe hybridised with a second type of target. A higher signal obtained for one of the first or second complex may be indicative, for example, of a higher degree of identity between the capture probe and the target which gives the highest signal.

In accordance with the present invention, the target may comprise, for example, DNA, RNA, or nucleic acid analogs (e.g. PNA (peptide nucleic acids), LNA (locked nucleic acids)) etc. More particularly, the target may comprise, for example, deoxyribonucleotides, ribonucleotides, modified deoxyribonucleotides (nucleotide or base analogs) or modified ribonucleotides (ribonucleotide or base analogs).

Similarly, the capture probe may also comprise, for example, DNA, RNA, nucleic acid analogs (e.g. PNA (peptide nucleic acids), LNA (locked nucleic acids)). The capture probe may therefore comprise deoxyribonucleotides, ribonucleotides, modified deoxyribonucleotides or modified ribonucleotides.

Suitable nucleotide or base analogs includes for example, 2′-deoxyInosine (dI or inosine), dideoxyribonucleotides (ddNTPs), 7-deaza-8-aza-G, phosphorothioate nucleic acids, peptide nucleic acids (PNA), locked nucleic acids (LNA), (3-2)-a-L-threose nucleic acids (TNA), 5-bromo-2-deoxyuridine (BrdU), 2,6-diaminopurine, deoxyribonucleotide triphosphate (dDapTP), 5-iodocytosine deoxyribonucleoside triphosphate (IdCTP), 5-bromo-uracil, 5-methyl-cytosine, 5-bromocytosine, 3-methyl 7-propynyl isocarbostyril nucleoside, 3-methyl isocarbostyril nucleoside, 5-methyl isocarbo-styril nucleoside, 7-nitroindole 2′-deoxyribonucleoside d(7-Ni), Iso-dC and Iso-dG.

The method of the present invention may use, for example, a solid support which is made from a material that is able to bind nucleic acids or analogs. The solid support may be selected, for example, from the group consisting of glass, plastic, silicon, gold particles, beads (microspheres), membranes, dextran, gels, etc. The capture probe may be part of a microarray.

The surface chemistry of the solid support may be modified with a chemical functional group able to allow association of the capture probe with the support. The surface may be modified, for example, by generating or grafting amine, aldehyde, or epoxy moieties. Probes and surfaces may also be modified by the grafting of spacers or linkers of various compositions, lengths, and structures (e.g. dendrimeric structures, grafting to poly-L-lysine films on glass, in situ DNA synthesis via photolithography). Probes may be spotted using an arrayer or any other technique known in the art. After spotting, the slides may be prepared for hybridisation experiments using standard procedures known to those skilled in the art (see Example 1). For example, when capture probes comprises DNA bound to a glass slide, an aldehyde coating may be used.

In accordance with the present invention, the method of detection used herein may be a passive hybridisation method or an active hybridisation method (e.g. flow-through hybridisation using active mass transport such as microfluidic or fluidic systems). For example, hybridisation may be carried out in a passive chamber and microarrays may be scanned and analysed using confocal microscopy (see Example 1).

Examples of target detection using methods and probes of the present invention are given herein. The examples of probes and targets etc. mentioned herein are not intended to be restrictive, i.e., other target such as fragments generated by enzymatic restriction or other amplicons or probes of other sequences may suitably be used without departing from the scope of the invention.

The present invention also relates in an aspect thereof, to the detection of the ermB gene of Staphylococcus aureus. For example, methods for the detection of a PCR amplicon of the ermB gene are encompassed herewith. The method may be useful for example, to the diagnosis of an infection of an individual with S. aureus and also for the determination of the antibiotic resistance profile of the bacteria.

In accordance with the present invention, a PCR amplicon generated from the ermB gene may be, for example, (inclusively) 550 nucleotides long or less (e.g., 450 nucleotides long or less, etc).

For example, the ermB capture probe may bind to a region located between nucleotide no. 1 and nucleotide no. 220 or between a region located between nucleotide no. 330 and nucleotide no. 550 of a PCR amplicon of 550 nucleotides long.

More particularly, according to the present invention, when the capture probe are designed to bind to a region located between nucleotide no. 1 and nucleotide no. 220 of the target, the capture probe may be linked to the support by a probe 5′ end. Additionally, when the capture probe binds to a region located between nucleotide no. 330 and nucleotide no. 550 of the target, the capture probe may be linked to the support by a probe 3′ end. In these particular examples, the unhybridised portion of the target which extends away (i.e., overhang) from the solid support is 220 nucleotides long or less.

In accordance with the present invention, a ermB PCR amplicon may be generated by standard PCR or by asymmetrical PCR using a primer pair selected, for example, from the group consisting of a primer pairs comprising SEQ ID NO.: 1, SEQ ID NO.: 2, SEQ ID NO.: 3 and SEQ ID NO.: 4 (and including primers consisting of SEQ ID NO.: 1, SEQ ID NO.: 2, SEQ ID NO.: 3 or SEQ ID NO.: 4). The capture probe may thus comprise a sequence which may be selected from the group consisting of SEQ ID NO.:14, SEQ ID NO.:15, SEQ ID NO.:16, SEQ ID NO.:17 and analogs thereof or any other probe which is able to bind a portion of the target located in the desired region.

Of course, any of the primer pair mentioned herein will be selected so that at least one of the primers may bind to a sense strand of the target and one of the primers may bind to an anti-sense strand of the same target.

Further in accordance with the present invention, the ermB capture probe may also comprise a sequence selected from the group consisting of SEQ ID NO.:13, SEQ ID NO.:14, SEQ ID NO.:15, SEQ ID NO.:16, SEQ ID NO.:17, SEQ ID NO.:18 (and including primers consisting of SEQ ID NO.:13, SEQ ID NO.:14, SEQ ID NO.:15, SEQ ID NO.:16, SEQ ID NO.:17, SEQ ID NO.:18) and analogs. In such instance and in accordance with the present invention, the target may be selected so that the probe binds a region located between nucleotide no. 1 and nucleotide no. n or between nucleotide no. m and nucleotide no. q of the target.

The present invention relates in a further aspect thereof, to the detection of the tuf gene of a Staphylococcus species. For example, the Staphylococcus species may be Staphylococcus hominis. Staphylococcus hominis may be obtained from the ATCC under no. 27844. The method may be useful for example, in the diagnosis of an infection of an individual with S. hominis.

In accordance with the present invention, the PCR amplicon generated from the tuf gene may be, for example, 600 nucleotides long or less (e.g., 550 nucleotides long or less, etc).

In accordance with the present invention the tuf capture probe may bind to a region located between nucleotide no. 1 and nucleotide no. 240 or a region located between nucleotide no. 360 and nucleotide no. 600 of a PCR amplicon of 600 nucleotides long or less.

More particularly, when the capture probe binds to a region located between nucleotide no. 1 and nucleotide no. 240 of the target, the capture probe may be linked to the support by the probe's 5′ end. Additionally, when the capture probe binds to a region located between nucleotide no. 360 and nucleotide no. 600 of the target, the capture probe may be linked to the support by the probe's 3′ end. In these specific examples, the unhybridised portion of the target which extends away (overhang) from a solid support is 240 nucleotides long or less.

In accordance with the present invention, a tuf PCR amplicon may be generated by standard PCR or by asymmetrical PCR using a primer pair selected, for example, from the group consisting of a primer pair comprising SEQ ID NO.: 5, SEQ ID NO.: 6 (including primers consisting of SEQ ID NO.: 5 or SEQ ID NO.: 6) and analogs thereof. In such cases, the capture probe may comprise SEQ ID NO.:19 or an analog thereof or any other probe which is able to bind a portion of the target located in the desired region.

Further in accordance with the present invention, the tuf capture probe may also comprise a sequence selected from the group consisting of SEQ ID NO.:19, SEQ ID NO.:20, SEQ ID NO.:21, SEQ ID NO.:22 (including probes consisting of SEQ ID NO.: 19, SEQ ID NO.:20, SEQ ID NO.:21, or SEQ ID NO.:22) and analogs thereof. In such instance and in accordance with the present invention, the target may therefore be selected so that the probe may bind a region located between nucleotide no. 1 and nucleotide no. n or between nucleotide no. m and nucleotide no. q of the target.

The present invention also relates in an additional aspect to detection of the bla_{SHV} gene of Escherichia coli, for example, E. coli strain CCRI-1192. The method may be therefore particularly useful in the diagnosis of an infection of an individual with E. coli and also in the determination of the antibiotic resistance profile of the bacteria.

In accordance with the present invention, the PCR amplicon generated from the bla_{SHV} gene gene may be, for example, (inclusively) 1000 nucleotides long or less, 800 nucleotides long or less, etc.

In accordance with the present invention, when such amplicon is used, the bla_{SHV} capture probe may bind to a region located between nucleotide no. 1 and nucleotide no. 400 or between a region located between nucleotide no. 600 and nucleotide no. 1000 of a PCR amplicon of 1000 nucleotides long.

More particularly, the bla_{SHV} capture probe binds to a region located between nucleotide no. 1 and nucleotide no. 400 of the target, the capture probe may be linked to the support by a probe 5′ end thereof. Additionally and in accordance with the present invention, when the capture probe binds to a region located between nucleotide no. 600 and nucleotide no. 1000 of the target, the capture probe may be linked to the support by a probe 3′ end. In such specific examples, the unhybridised portion of the bla_{SHV} gene which extends away (overhang) from a solid support is 400 nucleotides long or less.

In accordance with the present invention, a bla_{SHV} PCR amplicon may be generated by standard PCR or by asymmetrical PCR using a primer pair selected, for example, from the group consisting of a primer pair comprising SEQ ID NO.: 7, SEQ ID NO.: 8, SEQ ID NO.: 9, SEQ ID NO.: 10, SEQ ID NO.: 11, SEQ ID NO.: 12, and analogs thereof. In such cases, the capture probe may comprise SEQ ID NO.:23 or an analog thereof or any other probe which is able to bind a portion of the target located in the desired region.

In a further aspect, the present invention relates to a method for increasing the efficiency of detection of a nucleic acid-based target. The method may comprise contacting the target with a solid support-anchored oligonucleotide-based capture probe (e.g. single-stranded). The probe may be substantially complementary to a portion of a region located between nucleotide no. 1 and nucleotide no. n or between nucleotide no. m and nucleotide no. q of the target,
wherein n may be defined according to the formula n=0.4q,
wherein m may be defined according to the formula m=0.6q,
wherein q is the total nucleotide number of the target,
wherein when the capture probe is binding a region located between nucleotide no. 1 and nucleotide no. n of the target, the capture probe may be anchored to the solid support by its 5′ end thereof,
wherein when the capture probe is binding a region located between nucleotide no. m and nucleotide no. q of the target, the capture probe may be anchored to the solid support by its 3′ end thereof, and
wherein the capture probe generates a higher (e.g., more intense) signal in comparison to a signal measured for a second capture probe which binds to a region outside of the desired region (i.e., a region located between nucleotide no. 1 and nucleotide no. n or between nucleotide no. m and nucleotide no. q of the target).

The target to which the present method may be applied, encompass, for example, a target which, following binding (hydridisation) to the probe, has an unhybridised portion susceptible of being in contact with a substantially complementary sequence. Such as for example, the complementary strand or a double-stranded target.

In accordance with the present invention, the method may further comprise a step of detecting a complex formed by a (hybridized) capture probe and target.

In accordance with the present invention, the signal intensity measured for target bound to the capture probe of the present invention is higher than a signal intensity measured for a target (similar or the same) which hybridizes with another probe located outside of the region.

In accordance with the present invention, the closer the region of the target to which the probe binds is to nucleotide no. 1 or nucleotide no. q of the target, the higher may be the signal obtained.

The method of increasing the detection of targets of the present invention may be applied for example to a target which may contain 1000 nucleotides long and more and which may have following binding to the probe of the present invention, an unhybridised portion (overhang) of about 400 nucleotides long or less. Additionally, the method may be applied to a target which may comprise 625 nucleotides and more and which may have an unhybridised portion (overhang) of about 250 nucleotides long or less. Alternatively, the method of the present invention may be applied to a target which may comprise 400 nucleotides and more and which may have an unhybridised portion (overhang) of about 150 nucleotides long or less. Also alternatively, the method of the present invention may be applied to a target which may comprise 150 nucleotides and more and which may have an unhybridised portion (overhang) of about 60 nucleotides long or less.

The sequence of the genes mentioned herein may be found, for example, at the following GenBank accession numbers: AF239773 for the gene ermB, AF298802 for the gene tuf; and AF124984 for the gene bla_{SHV}. Theses sequences as well as any other mentioned herein are incorporated herein by reference.

In yet a further aspect, the present invention relates to an oligonucleotide-based capture probe for detection of a nucleic acid-based target, the capture probe may be able to bind to a substantially complementary target nucleotide sequence, whereby upon hybridisation of the capture probe and the target, a length (in number of nucleotides) of an unhybridised portion of the target which extends away from a solid support to which the capture probe is anchored, may be about 40% or less of the total length (in number of nucleotides) of the target.

In accordance with the present invention, the probe may be, for example, single-stranded.

Also in accordance with the present invention, the probe may be generated in situ.

Further in accordance with the present invention, the capture probe may be for example, from about 10 to about 70 nucleotides long, such as for example, from about 10 to about 50 nucleotides long, or for example, from about 10 to about 30 nucleotides long or from about 10 to about 25 nucleotides long.

In accordance with the present invention, the capture probe may be anchored to the support by its 5′ end and may be substantially complementary to a nucleotide sequence of the target that is located (inclusively) between nucleotide no. 1 and nucleotide no. n,
wherein n is defined according to the formula n=0.4q, and
wherein q is the total nucleotide number of the target.

Further in accordance with the present invention, the capture probe may be anchored to the support by its 3′ end and may be substantially complementary to a nucleotide sequence of the target that is located between (inclusively) nucleotide no. m and nucleotide no. q of the target,
wherein m is defined according to the formula m=0.6q, and
wherein q is the total nucleotide number of the target.

Capture probes of the present invention may either bind to a sense strand of a target or to an anti-sense strand of a target.

In accordance with the present invention, the capture probe and the region to which it binds may be of the same length (size, number of nucleotides) or may substantially be of the same length (i.e., may be slightly longer or slightly shorter).

It is to be understood herein that capture probes which are able to bind (under conditions that promote hybridisation between the target and the probe) at least a portion of a first target and a portion of a second target, where the portions are less than 100% identical to one another are encompassed herein. A differential signal intensity may therefore be measured between the first and second target upon hybridisation with the capture probe thereof are also encompassed herewith.

In addition, a capture probe which has a higher percentage of complementary to the portion of the first target than to the portion of the second target may be used in methods of the present invention and are therefore, also encompassed herein. For example, the portion of the first target and the portion of the second target may be from about 40% to 99.99% identical (or similar) and will therefore bind to the probe with different ability.

The capture probe may further comprise a spacer and/or a linker at the extremity (either at the 5′ end or at the 3′ end) which is to be anchored.

Examples of capture probes of the present invention include, for example, those which may bind to the ermB gene of Staphylococcus aureus, such as for example a PCR amplicon generated from the ermB gene.

In accordance with the present invention, the ermB PCR amplicon which may be detected with capture probes of the invention may be about 550 nucleotides long or less, e.g., 450 nucleotides long or less, etc.

Capture probes which may bind to a region located between nucleotide no. 1 and nucleotide no. 220 or to a region located between nucleotide no. 330 and nucleotide no. 550 of such examplary ermB PCR amplicon are encompassed by the present invention.

When the ermB capture probe binds to a region located between nucleotide no. 1 and nucleotide no. 220 of the target, the capture probe is generally linked to the support by its 5′ end thereof.

In contrast, when the ermB capture probe binds to a region located between nucleotide no. 330 and nucleotide no. 550 of the target the capture probe generally linked to the support by its 3′ end thereof.

Additionally, ermB capture probes which upon hydridisation with the ermB PCR amplicon leave, an unhybridised portion which extends away from a solid support of less than 220 nucleotides long, are encompassed by the present invention.

For example, PCR amplicons generated with a primer pair selected from the group consisting of a primer pair comprising SEQ ID NO.: 1, SEQ ID NO.: 2, SEQ ID NO.: 3 and SEQ ID NO.: 4 are efficiently detected by the capture probes of the present invention.

For example, a capture probe which may comprise a sequence selected from the group consisting of SEQ ID NO.:14, SEQ ID NO.:15, SEQ ID NO.:16, SEQ ID NO.:17 and analogs thereof may suitably be used to detect ermB PCR amplicons referred herein.

Other ermB capture probes, including those which comprise a sequence selected from the group consisting of SEQ ID NO.:13, SEQ ID NO.:14, SEQ ID NO.:15, SEQ ID NO.:16, SEQ ID NO.:17, SEQ ID NO.:18 and analogs thereof, may be suitably used when the target is selected, for example, so that the probe is able to bind a region located between nucleotide no. 1 and nucleotide no. n or between nucleotide no. m and nucleotide no. q of the target.

Other examples of capture probes of the present invention include, for example, those which binds a tuf gene from a Staphylococcus species, such as Staphylococcus hominis, and including, for example a PCR amplicon generated from the tuf gene.

In accordance with the present invention, the tuf PCR amplicon which may be detected by methods of the present invention may be about 600 nucleotides long or less, e.g., 550 nucleotides long or less, etc.

Capture probes which may bind to a region located between nucleotide no. 1 and nucleotide no. 240 or between a region located between nucleotide no. 360 and nucleotide no. 600 of such exemplary tuf PCR amplicon are encompassed by the present invention.

When the tuf capture probe binds to a region located between nucleotide no. 1 and nucleotide no. 240 of the target, the capture probe is generally linked to the support by its 5′ end thereof.

In contrast, when the capture probe binds to a region located between nucleotide no. 360 and nucleotide no. 600 of the target the capture probe is generally linked to the support by its 3′ end thereof.

Additionally, tuf capture probes which upon hydridisation with the tuf PCR amplicon, leave an unhybridised portion which extends away from a solid support of less than 240 nucleotides long are encompassed by the present invention.

For example, tuf PCR amplicons generated with a primer pair selected from the group consisting of a primer pair comprising SEQ ID NO.: 5, SEQ ID NO.: 6 and analogs thereof are efficiently detected by the probes of the present invention.

For example, a capture probe which may comprise a sequence selected from the group consisting of SEQ ID NO.:19 or analogs thereof may suitably be used to detect tuf PCR amplicons referred herein.

Other tuf capture probes, including those which comprises a sequence selected from the group consisting of SEQ ID NO.:19, SEQ ID NO.:20, SEQ ID NO.:21, SEQ ID NO.:22 and analogs thereof, may be suitably used when the target is selected, for example, so that the probe is able to bind a region located between nucleotide no. 1 and nucleotide no. n or between nucleotide no. m and nucleotide no. q of the target.

Further examples of capture probes of the present invention include, for example, those which binds bla_{SHV} gene of Escherichia coli such as for example, the CCRI-1192 strain of E. coli. Targets encompassed by the present invention include a bla_{SHV} PCR amplicon.

In accordance with the present invention, the bla_{SHV} PCR amplicon which may be detected with methods of the present invention, may be about 1000 nucleotides long or less, e.g., 800 nucleotides long or less, etc.

Capture probes which may bind to a region located between nucleotide no. 1 and nucleotide no. 400 or between a region located between nucleotide no. 600 and nucleotide no. 1000 of such examplary bla_{SHV} PCR amplicon.

When the capture probe binds to a region located between nucleotide no. 1 and nucleotide no. 400 of the target, the capture probe is generally linked to the support by its 5′ end thereof.

In contrast, when the capture probe binds to a region located between nucleotide no. 600 and nucleotide no. 1000 of the target, the capture probe is generally linked to the support by its 3′ end thereof.

Additionally, bla_{SHV} capture probes which upon hydridisation with the bla_{SHV} PCR amplicon leave an unhybridised portion which extends away from a solid support of less than 400 nucleotides long are encompassed by the present invention.

For example, bla_{SHV} PCR amplicons generated with a primer pair selected from the group consisting of a primer pair comprising SEQ ID NO.: 7, SEQ ID NO.: 8, SEQ ID NO.: 9, SEQ ID NO.: 10, SEQ ID NO.: 11, SEQ ID NO.: 12, and analogs thereof are efficiently detected by the probes of the present invention.

For example, a capture probe which may comprise a sequence selected from the group consisting of SEQ ID NO.: 23 or analogs thereof may suitably be used to detect bla_{SHV} PCR amplicons referred herein.

In a further aspect, the present invention relates to probes, arrays and kits comprising the sequences defined herein.

In an additional aspect, the present invention provides an array comprising at least one capture probe of the present invention.

In yet an additional aspect, the present invention provides kits comprising at least one capture probe of the present invention.

Probes which have been selected by methods of the present invention may be used with various hybridisation reagents, buffers and conditions. For example, probes and detection methods of the present invention may suitably be used in combination with hybridisation facilitators which may enhance hybridisation kinetics (e.g. betaine, formamide, tetramethyl ammonium chloride (TMAC)) or other reagents which may be used to reduce the hybridisation time and/or increase the sensitivity of the reactions required for detection of hybrids (a probe/target complex).

The capture probe and method of the present invention are to be used for detection of a nucleic acid-based target from a pluricellular organism which may be present, for example, in heterogenous forms (i.e., varies from an organism to another or from a gene of an organism to another).

The capture probe and method of the present invention may also be used for detection of a nucleic acid-based target from a microorganism (e.g. algae, bacteria, archaea, virus, fungi, yeast or parasite), which may be present, for example, in heterogenous forms (i.e., varies from an organism to another or from a gene of an organism to another).

The capture probe of the present invention may also be used for epidemiological purposes such as strain typing or species (subspecies) typing.

The capture probe and method of the present invention may therefore be used for molecular diagnostic purposes, single nucleotide polymorphism detection, allelic heterogeneity determination, genotyping, isotyping, strain typing or epidemiological typing or in any methods which may require a higher level of sensitivity and a high discriminatory power.

The present invention relates in one aspect thereof to a method for designing an oligonucleotide-based capture probe for the detection of a nucleic acid-based target, the method may comprise:
identifying a region located between nucleotide no. 1 and nucleotide no. n or between nucleotide no. m and nucleotide no. q of the target, wherein n is defined according to the formula n=0.4q (i.e., (n/q)×100=40%), wherein m is defined according to the formula m=0.6q (i.e., (m/q)×100=60%), and wherein q is the total nucleotide number of the target and
providing a single-stranded oligonucleotide-based capture probe substantially complementary to a portion of the region,
whereby when the capture probe is binding a region located between nucleotide no. 1 and nucleotide no. n of the target, the capture probe is to be anchored to a solid support by its 5′ end thereof,
whereby when the capture probe is binding a region located between nucleotide no. m and nucleotide no. q of the target the capture probe is to be anchored to a solid support by a its 3′ end thereof and
whereby the target after binding to the probe has an unhybridised portion susceptible of being in contact with a substantially complementary sequence.

In accordance with the present invention the capture probe designed according to the present method may generate a higher signal in comparison to a signal measured for a second capture probe which binds to a target region outside of the region located between nucleotide no. 1 and nucleotide no. n or between nucleotide no. m and nucleotide no. q of the target.

In accordance with the present invention, the capture probe may be 100% complementary to the portion of the target to which it binds. Also in accordance with the present invention, the capture probe may be from 90% to 99.99% complementary of the target to which it binds. Additionally, the capture probe may be from 70% to 99.99% complementary to the portion.

A probe analog or variant is to be understood herein as having at least about 70% identity with a desired probe completer.

The expression “substantially complementary” is to be understood herein as referring to sequences which are complementary and which comprises at least about 70% of sequences being complementary to one another.

Further scope and applicability will become apparent from the detailed description given hereinafter. It should be understood however, that this detailed description, while indicating preferred embodiments of the invention, is given by way of illustration only, since various changes and modifications within the spirit and scope of the invention will become apparent to those skilled in the art.

It is to be understood herein, that if a “range” or “group of substances” is mentioned with respect to a particular characteristic (e.g., temperature, concentration, time and the like) of the present invention, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example:
with respect to a length of 1000 nucleotides long or less, is to be understood as specifically incorporating herein each and every individual length, e.g., a length of 999, 592, 585, 273, 129, 93, etc.; Therefore, unless specifically mentioned, every range mentioned herein is to be understood as being inclusive. For example, when a region is located between nucleotide no. 1 and nucleotide no. n, it is to be understood that the region includes nucleotide no. 1 and n. Similarly, an expression such as, “550 nucleotides long or less” includes a length of 550, etc.
with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;
and similarly with respect to other parameters such as concentrations, elements, etc. . . .

It is in particular to be understood herein that the sequences, regions, portions defined herein each include each and every individual sequences, regions, portions described thereby as well as each and every possible sub-sequences, sub-regions, sub-portions whether such sub-sequences, sub-regions, sub-portions is defined as positively including particular possibilities, as excluding particular possibilities or a combination thereof, for example an exclusionary definition for a region may read as follows: “provided that when said region is comprised between nucleotide no. X and nucleotide no. Y said probe may not be anchored by a probe's 3′ end”. Another example of a negative limitation is the following; provided that the target is no shorter than 50 nucleotides (i.e., is 50 nucleotides long or longer). Yet another example of a negative limitation is the following: provided that the length of the probe is no shorter than 10 nucleotides (i.e, is 10 nucleotides and longer). Yet a further example of a negative limitation is the following; a sequence comprising SEQ ID NO.: X with the exclusion of a gene encoding X.; etc.

It is also to be understood herein that “g” or “gm” is a reference to the gram weight unit and “C”, or “° C.” is a reference to the Celsius temperature unit.

### BRIEF DESCRIPTION OF THE DRAWINGS

Having thus generally described the invention, reference will be made to the accompanying drawings, showing by way of illustration only an illustrative embodiment thereof and in which:

FIG. 1 shows the position of capture probes and PCR primers on the ErmB gene PCR amplicons of either 402 base pairs (bp) (Panel A) or 433 bp (Panel B). Arrows represent primers used for generating these amplicons. Dashed boxes represent 5′ amino-modified probes. Brackets indicate the length of the 5′ overhanging tail (overhang) of the target strand captured by each capture probe,

FIG. 2 illustrates the correlation between intensity of the fluorescence signal for 16 hours hybridisations and the length of the 5′ overhang of the captured ermB amplicon strand. Panel A shows results for capture probes A-S-ErmBH272 and A-S-ErmBH272a hybridising to both ermB amplicons (i.e. 402- and 433-bp amplicons). Panel B shows hybridisation of probes A-S-ErmBH370 and A-S-ErmBH370a also hybridising to both ermB amplicons. Panel C shows hybridisation of probes A-S-ErmBH459 and A-S-ErmBH459a hybridising to both ermB amplicons. For all panels, each value represents the mean of three replicates. The standard deviation for these replicates is also shown,

FIG. 3 illustrates the hybridisation kinetics for the six oligonucleotide capture probes targeting ErmB. Arrays were hybridised for 15, 30, 60, 180 and 960 minutes (16 hours) to the denatured double-stranded 433-bp ermB amplicon. (Panel A) Hybridisation to capture probe A-S-ErmBH272a (164 nucleotides from the 5′ end). (Panel B) Hybridisation to capture probe A-S-ErmBH370 (151 nucleotides from the 5′ end). (Panel C) Hybridisation to capture probe A-S-ErmBH459 (62 nucleotides from the 5′ end). (Panel D) Hybridisation to capture probe A-S-ErmBH272 (249 nucleotides from the 5′ end). (Panel E) Hybridisation to capture probe A-S-ErmBH370a (262 nucleotides from the 5′ end). (Panel F) Hybridisation to capture probe A-S-ErmBH459a (351 nucleotides from the 5′ end). For all panels, each value is the mean of three replicates. The standard deviation for these replicates is also shown. The scale for the fluorescence intensity axis is different for each panel to better illustrate the shape of the graphs. The letter (A) or (B) attributed beside each of the tested probe refers to amplicons generated with PCR primers of FIG. 1A or FIG. 1B, respectively,

FIG. 4 illustrates idealised interactions between an immobilised DNA probe and the two strands of the target amplicon. The target strand (T*) hybridises to the DNA probe, leaving a 5′ overhang of variable length depending on the location of the region of the captured amplicon strand targeted by the probe. (Panel A) T* hybridised to the DNA probe, leaving a long 5′ overhang of the captured product strand targeted by the probe. (Panel B) T* hybridised to the DNA probe, leaving a short 5′ overhang of the captured product strand targeted by the probe. (Panel C) The free complementary strand (T′) of the target product hybridised to the overhanging tail of T*, generating a branch migration that caused destabilisation of the secondary complex. (Panel D) The free T* (T*free) hybridised to the free region of T′, generating an antagonistic branch migration that prevented the first branch migration from breaking the secondary complex,

FIG. 5 illustrates hybridisation to a microarray of capture probes of single-stranded target amplicon strand (T*) generated by asymmetrical PCR followed by hybridisation with the complementary amplicon strand (T′). T* was hybridised for 10 h to the ermB array. Non-hybridised T* (T*free) was then washed away, and the array was hybridised another 16 h with an equimolar quantity of the complementary strand T′ (grey boxes) or with hybridisation buffer only (black boxes). Slides were washed prior to fluorescence detection. A significant decrease in signal intensity was observed when the complementary strand T′ was hybridised for 16 hours compared to the control hybridisation using buffer only. (Panel A) Hybridisation to the lower (anti-sense or non-coding) strand of the 433-bp ermB amplicon followed by hybridisation with the upper (sense) strand of the same amplicon. (Panel B) Hybridisation to the upper strand of the 433-bp amplicon followed by hybridisation to the lower strand of the same amplicon. For both panels, each result is the mean of three replicates,

FIG. 6 shows the correlation between the fluorescence intensity and the length of the 5′ overhang of captured tuf probes hybridised to different area of the 523-bp tuf PCR product amplified from Staphylococcus hominis. Probes A-S-TShoH520 (complementary to the lower strand) and A-S-TShoH520a (complementary to the upper strand) target the same region of the S. hominis product. Each value is the mean of three replicates. Standard deviation for these replicates is also shown,

FIG. 7 shows the correlation between the fluorescence intensity and the length of the 5′ overhang of the captured blaSHV probe A-S-Shv1H691 hybridised to different blaSHV products of 182 to 715 bp. Each value is the mean of three replicates. Standard deviation for these replicates is also shown,

FIG. 8 shows the position of capture probes and PCR primers on the tuf gene PCR amplicons of 523 bp. Arrows represent primers while dashed boxes represent 5′ amino-modified probes. Brackets indicate the length in nucleotides of the 5′ overhanging tail of the target strand captured by each capture probe, and;

FIG. 9 shows the position of PCR primers and a capture probe on the blaSHV gene PCR amplicons of 182 to 715 bp. Arrows represent primers used for generating these amplicons. The single dashed box represents a 5′ amino-modified probe. Brackets indicate the length in nucleotides of the 5′ overhanging tail of the target strand captured by the capture probe for each different PCR amplicons generated.

Other objects, advantages and features of the present invention will become apparent upon reading of the following non-restrictive description of preferred embodiments with reference to the accompanying drawing which is exemplary and should not be interpreted as limiting the scope of the present invention.

### DETAILED DESCRIPTION OF THE INVENTION

The present invention is illustrated in further details by the following non-limiting examples.

### EXAMPLES

### Example 1

Correlation between the efficiency of microarray DNA hybridisation and the length of the 5′ overhang of captured ermB amplicon strands.

Materials and Methods

Microarray Production

Twenty-mer oligonucleotide probes bearing a 5′ amino-linker were synthesised by Biosearch Technologies (Novato, Calif., USA). Capture probe sequences used in the present invention are described in Table 1. The amino linker modification allowed covalent attachment of probes onto aldehyde-coated glass slides (CEL Associates, Pearland, Tex., USA). Oligonucleotide probes were diluted 2-fold in ArrayIt™ MicroSpotting Solution Plus (Telechem International, Sunnyvale, Calif., USA) to a final concentration of 5 μM. Oligonucleotides were spotted in triplicate using a VIRTEK SDDC-2 arrayer (Bio-Rad Laboratories, Hercules, Calif., USA) with SMP3 pins from Telechem International. After spotting, slides were dried overnight, washed by immersion in 0.2% sodium dodecyl sulfate (SDS; Laboratoire Mat, Quebec, QC, Canada) for 2 min, and rinsed in ultrapure water for 2 min. Slides were boiled in ultrapure water for 5 min for washing out the unbound oligonucleotides. Imine bonds between the glass surface and probes were reduced to a stable amide link by immersion for 20 min into a sodium borohydride solution (1 g sodium borohydride; Sigma, St. Louis, Mo., USA), 300 mL phosphate-buffered saline (PBS; also from Sigma), and 100 mL ethanol. Slides were then washed in 0.2% SDS for 1 min and rinsed in ultrapure water for 1 min. Slides were finally dried by centrifugation for 5 min under vacuum with a Savant SpeedVac™ Plus (Thermo Savant, N.Y., USA) and stored in a dry oxygen-free and dark environment. All above chemical treatments of the slides were performed at room temperature.

PCR amplification and amplicon labelling

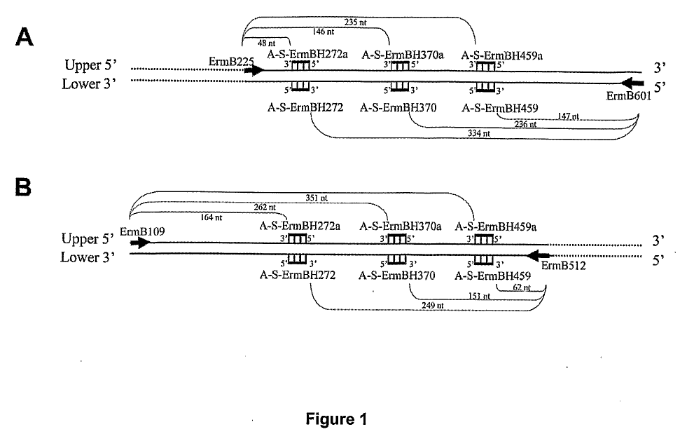

Fluorescent dyes (label) were incorporated during PCR amplification. Cy3 or Cy5 dUTP (Amersham Biosciences, Baie d'Urfé, QC, Canada) were mixed at concentrations of 0.02 μM in a 50-μL PCR mixture containing 0.05 mM dATP, 0.05 mM dCTP, 0.05 mM dGTP, 0.02 mM dTTP, 5 mM KCl, 1 mM Tris-HCl (pH 9.0), 0.01% Triton X-100, 2.5 mM MgCl2, 0.5 unit of Taq DNA polymerase (Promega, Madison, Wis., USA), 1 ng purified genomic DNA, and 0.2 μM of each of the two primers. To test the effect of oligonucleotide probe position on the captured target DNA strand on hybridisation efficiency, we amplified by PCR two overlapping portions (402 and 433 bp) of the Staphylococcus aureus ermB gene (FIG. 1). The ermB gene was amplified from genomic DNA isolated from the erythromycin-resistant S. aureus strain CCRI-1277. The 402-bp product was produced using primers ErmB225 and ErmB601, while the 433-bp product was amplified by PCR using primers ErmB109 and ErmB512 (Table 1). Thermal cycling for PCR amplification (180 s at 94° C., followed by 40 cycles of 5 s at 95° C., 30 at 55° C., and 30 s at 72° C.) was carried out on an MJ Research PTC-200 DNA Engine® thermal cycler (Bio-Rad Laboratories). PCR products were purified using the QIAquick® PCR purification kit (Qiagen, Mississauga, ON, Canada). The dye incorporation was measured with an Ultrospec 2000 Spectrophotometer (Amersham Biosciences) at 550 nm for Cy3 and at 650 nm for Cy5. Concentration of the amplified product was determined at 260 nm using the Ultrospec 2000.

Asymmetric PCR was performed using the PCR conditions described above, except that the upper strand of the 433-bp product was obtained using a 20:1 ratio of ErmB109 and ErmB512 primers, respectively (FIG. 1). An asymmetrical PCR was performed to produce the lower strand using a 20:1 ratio of ErmB512 and ErmB109, respectively (FIG. 1). Each asymmetric PCR was verified on a 1.5% agarose gel to ensure the production of single-stranded DNA and quantified using the Ultrospec 2000 at 260 nm. The concentration of single-stranded DNA was adjusted to 1 pM and hybridised to the microarray to confirm the absence of the complementary strand.

DNA microarray hybridisation and data acquisition

Prehybridisation and hybridisation were performed in 15×13 mm HybriWell™ self-sticking hybridisation chambers (Grace Bio-Labs, Bend, Oreg., USA). Microarrays were first prehybridised for 30 min at room temperature with 1× hybridisation solution (6× standard saline phosphate-EDTA [SSPE; EM Science, Gibbstown, N.J., USA], 1% bovine serum albumin [BSA], 0.01% polyvinylpyrrolidone [PVP], 0.01% SDS, and 25% formamide [all from Sigma]). Cy-dUTP-labeled PCR products were denatured at 95° C. for 5 min and then quickly chilled on ice. Five microliters of denatured labeled products were mixed with 10 μL of 2× hybridisation buffer (12×SSPE, 2% BSA, 0.02% PVP, and 0.02% SDS) and 5 μL formamide (final concentration of 25%). Prehybridisation solution was removed from the chamber and replaced by the labeled PCR products resuspended in hybridisation solution. The hybridisation was carried out at 22° C. for 15 min and up to 16 h. After hybridisation, microarrays were washed with 2×SSPE containing 0.1% SDS for 5 min at room temperature and rinsed once with 2×SSPE for 5 min. Microarrays were dried by centrifugation at 1350×g for 3 min. Slides were scanned using a ScanArray® 4000XL confocal scanner (Packard Bioscience Biochip Technologies, Billerica, Mass., USA), and fluorescent signals were analyzed using its software.

Results

We tested whether the region of the product targeted by an oligonucleotide capture probe influenced hybridisation efficiency. To achieve this goal, we initially used the ermB bacterial antibiotic resistance gene as genetic target. This gene encodes an adenine N-6-methyltransferase, which confers resistance to macrolides, lincosamides, and streptogramin B (Roberts et al., 1999, Antimicrob. Agents Chemother., 43:2823-2830). We generated two overlapping ermB PCR products, each targeted by six 20-mer capture probes located at different areas of the products (FIG. 1). Three of these probes (A-S-ErmBH272, A-S-ErmBH370, and A-S-ErmBH459) were designed to be complementary to the lower strand of both products, while the three other probes (A-S-ErmBH272a, A-S-ErmBH370a, and A-S-ErmBH459a) targeted the same region but hybridised to the upper strand of both products. For these perfectly complementary oligonucleotides, both strands have the same Tm and secondary structure, and have also been shown to behave identically for hybridisation in solution (Rafalski, 1988, Anal. Biochem., 173:383-386). Therefore, variations in the performance of hybridisation between capture probes targeting the same region located on the opposite strand of a product may be attributed to a bias correlated with the efficiency of hybridisation onto solid support.

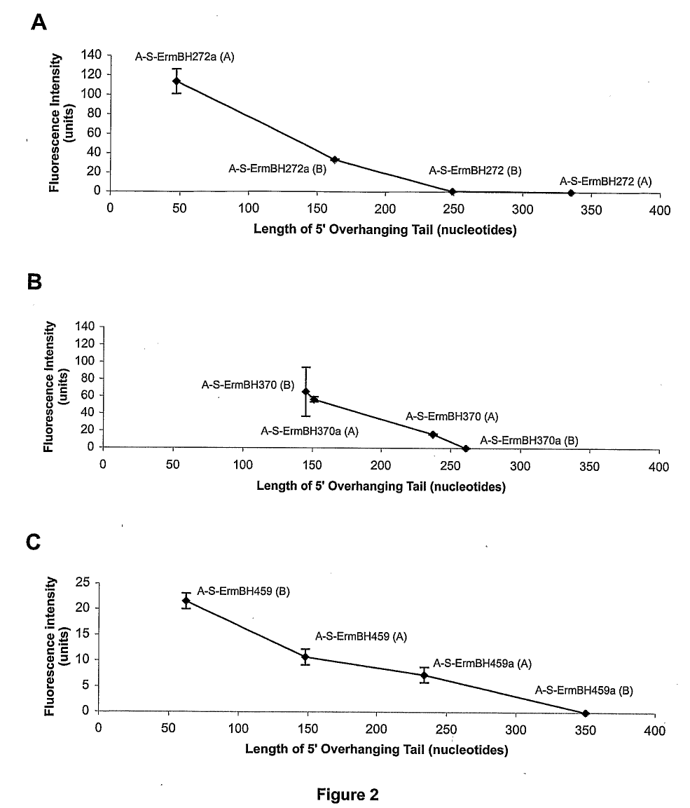

The Cy3-labeled 402- and 433-bp products were hybridised overnight to the ermB array that contained the six different capture probes (FIG. 1). After washing and analysis, it was observed that the fluorescence signal for each capture probe after a 16 hours hybridisation was not identical. Plotting the fluorescence intensities of hybridisation against the regions of the product recognized by capture probes revealed a correlation between the fluorescence intensity and the length of the free 5′ overhanging portion of the captured strand (FIG. 2). For each of the six capture probes, the strongest hybridisation signal was always observed for the probe targeting a region closest to the 5′ end of the upper or lower targeted strand. These probes hybridised the closest to the 5′ end of the complementary strand of the product, thus leaving the shortest overhanging 5′ end. Both target ermB products (402- and 433-bp) behaved similarly with respect to fluorescence intensity and position of the capture probe. Also, no significant difference was observed between the upper and lower strands. This is illustrated in FIG. 2B by hybridisation with oligonucleotides A-S-ErmBH370 of the 433-bp product which is 151 nucleotides from the 5′ end, and A-S-ErmBH370a of the 402-bp product which is 146 nucleotides from the 5′ end, showing that when the 5′ overhang lengths were similar, the fluorescence intensities were also similar regardless of the product size or the target strand.

Despite the fact that for the same oligonucleotide capture probe the key determinant for hybridisation intensity appears to be the length of the 5′ overhang of the hybridised target DNA strand, some probes worked better than others. For example, probe A-S-ErmBH272a (5′ overhang length of 48 nucleotides) produced a hybridisation signal six times stronger than probe A-S-ErmBH459 (5′ overhang length of 62 nucleotides). One explanation may be that the area covered by probe A-S-ErmBH459 may be less available for hybridisation or less stable once hybridised than the area covered by probes A-S-ErmBH272 and A-S-ErmBH272a (FIG. 2). This behavior may be attributed either to the secondary structure of the target strand or to thermodynamic properties of the probes. It is salient to point out that the AG of the secondary structure from probe A-S-ErmBH459 is −14.2 kcal/mol, which represents a much higher energy than that for the other probes used in this study (i.e. −5.3 kcal/mol for probe A-S-ErmBH272 and −3.5 kcal/mol for probe A-S-ErmBH370). Nonetheless, even if probe A-S-ErmBH459 gave a lower hybridisation signal, its intensity correlated with the length of the 5′ overhang (FIG. 2 C).

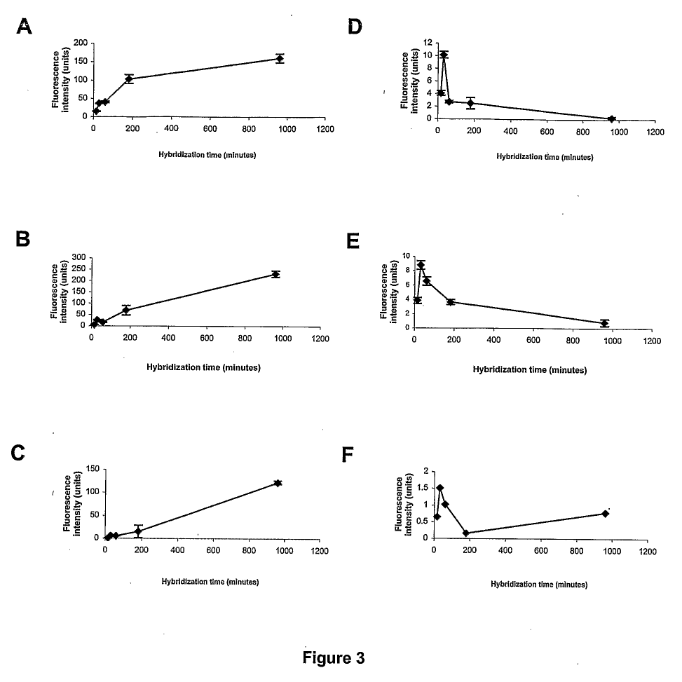

Thus, capture probes (P) targeting (able to bind) the 5′ end of the captured target strand (T*) gave strong and reproducible hybridisation signals, while probes targeting (able to bind) the 3′ extremity of the captured target strand gave no or very weak hybridisation signals after overnight hybridisation. One plausible explanation is that T* hybridised by its 3′ end is less stable than the same strand hybridised closer to its 5′ end. To verify this hypothesis, hybridisation kinetics were assessed by hybridising the 433-bp labeled products with the ermB array for 15, 30, 60, 180 and 960 min (16 h). Probes targeting regions close to the 5′ end of either strand of the product showed a fluorescent signal increasing with hybridisation time (FIG. 3, Panels A, B and C). Probes targeting regions leaving a longer 5′ overhang of either strand of the products exhibited very different hybridisation kinetics (FIG. 3, Panels D, E and F). Indeed, we observed an increase of the hybridisation signal in the first 30 min of hybridisation, but thereafter fluorescence intensity decreased over time until it reached background levels. This kinetics of hybridisation during the first 30 minutes is also observed for probes targeting the 5′ end of the captured strand. It may be surmised that during the first 30 minutes of the reaction, local higher concentration of capture probe (P) favoured hybridisation of T* on P. This hybridisation behaviour appears to follow a classical equilibrium equation:

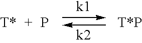

where k1 is the hybridisation constant and k2 the dissociation constant. This hybridization kinetics suggests that the longer the hybridisation period the more important is the negative impact of a long 5′ overhang.

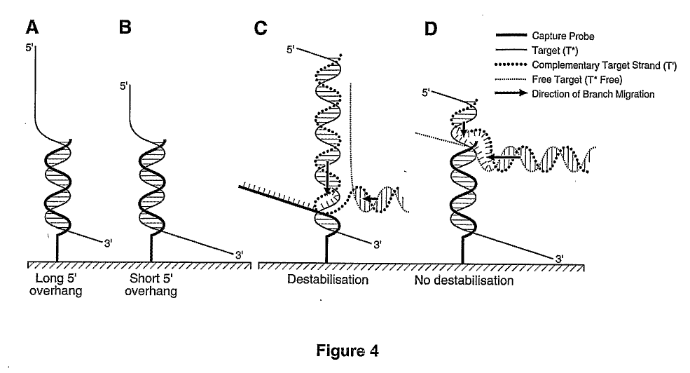

The hybridisation kinetics following the first 30 minutes, which is dependent on the position of the probe on the captured strand, may be explained by the topology of the T*P duplex. When a probe recognises an area closer to the 3′ end of the captured target strand T*, most of the overhanging 5′ end of non hybridised DNA is exposed to the liquid phase above the glass surface (FIG. 4A). On the other hand, when it hybridises to an area close to the 5′ end of the captured strand target, most of T* (3′ end) is directed towards the glass surface (FIG. 4B). In the first conformation, the overhanging tail of T* may be available for reassociation with its complementary strand; T′, a process that may destabilises the probe-target duplex (T*P).

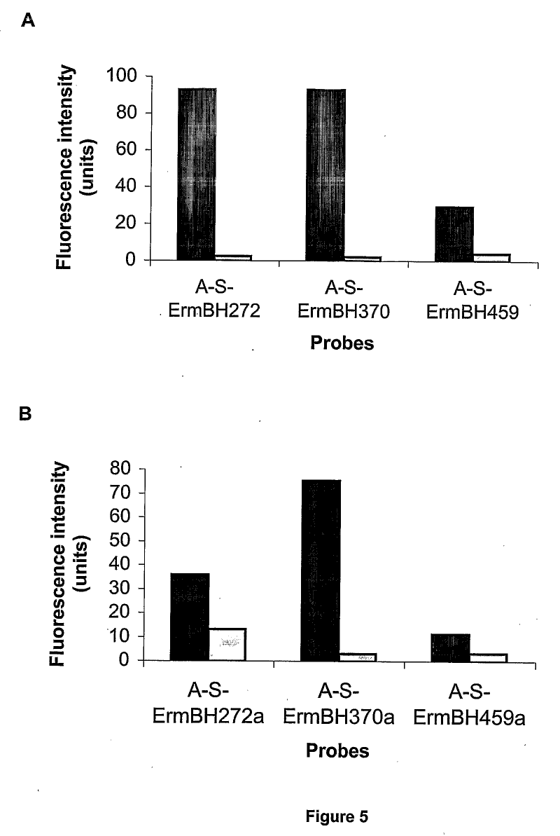

To test the ability of the nonhybridised complementary strand (T′) to destabilise the T*P duplex, we carried out experiments with single-stranded products. Microarrays were hybridised for 10 h with the amplified 433-bp ermB product lower strand (T*) generated by asymmetrical PCR. After washing out the nonhybridised T* still in solution (T*free), the hybridisation was carried out for an additional 16 h, either with hybridisation buffer only or with an equimolar amount of the complementary upper strand T′. In the presence of only single-stranded target DNAs (T*), the region at which the oligonucleotide probe hybridises no longer influences the hybridisation intensity (FIG. 5). For example, probe A-S-ErmBH272, which leaves a 5′ overhang of 249 nucleotides, hardly captures any of the target DNA when the double-stranded product is used as target (FIG. 2A). However, this same probe efficiently captured the complementary single-stranded DNA produced by asymmetrical PCR (FIG. 5A). Similar results were observed for hybridisation with the upper product strand. The intensity of fluorescence decreased dramatically when the complementary T′ lower (anti-sense) strand was included in the assay (FIG. 5B). The addition of the complementary strand T′ reduced the intensity of hybridisation close to background levels, suggesting that T*P duplex destabilisation occurs in the presence of the complementary strand. Displacement of T* from P by reassociation with T′ probably proceeds through a sequential displacement pathway also known as a zipper effect (Reynaldo et al., 2000, J. Mol. Biol., 297:511-520). Hybridisation between the captured T* strand and its complementary strand T′ in solution will occur first at the exposed overhang tail of the captured T* and will be followed by a branch migration mechanism towards the 3′ end. Such a mechanism was used to build a DNA-fuelled nanomolecular machine (Yurke et al., 2000, Nature, 406: 605-608; Alberti et al., 2003, Proc. Natl. Acad. Sci. USA, 100: 1569-1573). In those studies, the authors used the complementary DNA strand (called “fuel DNA”) to close and open double-stranded DNA structures. In the experiment described above, the complementary strand T′ seems to act as the “fuel” DNA, pulling the captured target strand T* from the probe (FIG. 4C). A longer 5′ overhang increases the probability of collision between the complex T*P and free T′ and thus leads to a faster destabilisation effect. This may explain the hybridisation bias observed with long 5′ overhangs but does not explain why a short 5′ overhang end generates a hybridisation signal that increases over time (FIG. 3 A, B, C).

### Example 2

Correlation between the efficiency of microarray DNA hybridisation and the length of the 5′ overhang of captured tuf amplicon strands.

Material and methods are the same as those used in Example 1 except that primers and capture probes targeting the tuf gene encoding the elongation factor Tu were used (see Table 1). The tuf gene was amplified from genomic DNA isolated from Staphylococcus hominis subsp. hominis strain ATCC 27844. A 523-bp product was produced using primers TshoH240 and TstaG765. Thermal cycling for PCR amplification was as described in Example 1.

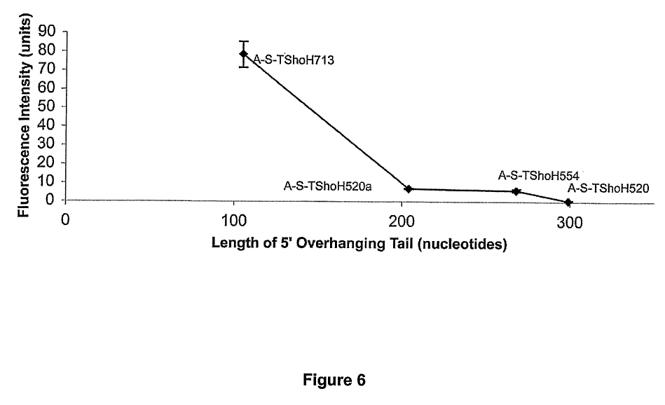

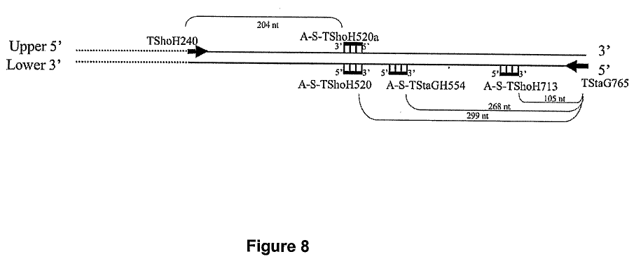

FIG. 8 shows the position of capture probes and PCR primers on the tuf gene PCR amplicons of 523 bp. Arrows represent primers while dashed boxes represent 5′ amino-modified probes. Brackets indicate the length in nucleotides of the 5′ overhanging tail of the target strand captured by each capture probe. Results with the tuf gene were similar to those obtained with ermB (FIG. 6). Capture probes gave stronger hybridisation signal when the 5′ overhanging tail was short and showed near background signals when the 5′ tail reached a length over 250 nucleotides for tuf (FIG. 6). Thus, different capture probes seem to follow similar hybridisation methods, irrespective of the target sequences.

To demonstrate that methods predicted in Example 1 are applicable to other DNA targets, we have tested the hybridisation efficiency of different capture probes (according to the region to which they hybridise) on the highly conserved tuf gene. As described in Example 1, capture probes gave stronger hybridisation signal when the 5′ overhang was short. In example 2, capture probes showed near background signals when the 5′ overhang reached a length over 250 nucleotides (FIG. 6).

### Example 3

Correlation between the efficiency of microarray DNA hybridisation and the length of the 5′ overhang of captured blaSHV amplicon strands.

Material and methods are the same as those used in Example 1 except that primers and capture probes targeting the blaSHV gene encoding a β-lactamase were used (see Table 1). The blaSHV gene was amplified from genomic DNA isolated from Escherichia coli strain CCRI-1192. Different products were generated by combining the reverse primer shv763 with five different primers used to produce different lengths of 5′ overhangs: (i) primer shv604 amplified a 182-bp product; (ii) primer shv449 amplified a 337-bp product; (iii) primer shv368 amplified a 418-bp product; (iv) primer shv313 amplified a 473-bp product; and (v) primer shvseq71 amplified a 715-bp product (Table 1). Thermal cycling for PCR amplification was as described in Example 1.

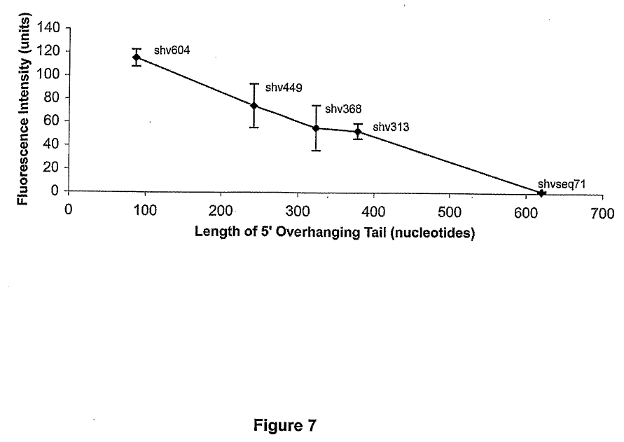

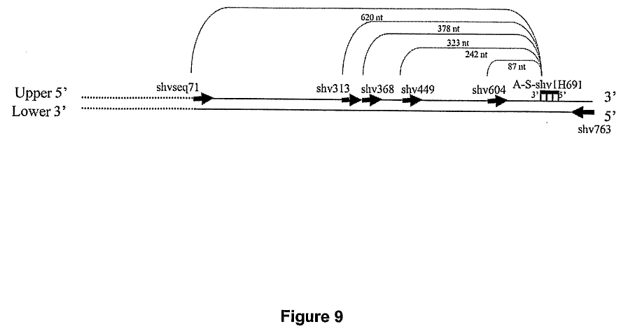

FIG. 9 shows the position of PCR primers and a capture probe on the blaSHV gene PCR amplicons of 182 to 715 bp. Arrows represent primers used for generating these amplicons. The single dashed box represents a 5′ amino-modified probe. Brackets indicate the length in nucleotides of the 5′ overhanging tail of the target strand captured by the capture probe for each different PCR amplicons generated. Results obtained with the blaSHV gene are shown in FIG. 7. Products were amplified using the same reverse primer but using different forward primers. This allowed the amplification of products having a variable forward length, while its reverse length remained constant. After hybridisation of each product to the microarray, we plotted the signal in function of the length of the 5′ tail for the probes targeting the upper strand and in function of the length of the 3′ tail for the probes targeting the lower strand. The increase of the length of the 5′ tail reduced the signal (correlation coefficient between −0.66 and −0.85), whereas the increase of the 3′ tail had no major effect on the hybridisation signal (correlation coefficient between 0.12 and 0.20) (FIG. 7). Those results suggest that, while the length of the 5′ tail has a significant impact on the hybridisation signal observed, the length of the 3′ tail seems less important (data not shown).

Therefore, the results for blaSHV were similar to those obtained with ermB in Example 1. Capture probes targeting blaSHV gave stronger hybridisation signal when the 5′ overhanging tail was short and showed near background signals when the 5′ end reached a length over 600 nucleotides for blaSHV (FIG. 7). Thus, different capture probes seem to follow similar hybridisation methods, irrespective of the target sequences.

The hybridisation behaviour and efficiency of oligonucleotides arrayed onto a solid support has been investigated herein. As described herein, we observed that the position of a capture probe on a given product has an impact on the observed hybridisation signal. The hybridisation behaviour of a double-stranded product DNA on short oligonucleotides immobilised by their 5′ end gave counter-intuitive and unexpected results. Indeed, one would assume weaker hybridisation signal when a 5′ end immobilised probe binds the target molecule close to its 5′ end, because of steric hindrance caused by a longer 3′ overhanging tail. However, our results show that the increase of the 3′ end has no major effect on hybridisation signal, whereas the hybridisation signal strength is inversely correlated with the length of the 5′ overhanging tail of the target molecule when hybridised with a probe immobilised via its 5′ end.

This hybridisation behavior may be explained by the topology of the T*P duplex. When a probe recognizes an area closer to the 3′ end of the captured target strand T*, most of the overhanging 5′ end of nonhybridised DNA is exposed to the liquid phase above the glass surface (FIG. 4). On the other hand, when it hybridises to an area close to the 5′ end of the captured strand target, most of T* (3′ end) is directed towards the glass surface. In the first conformation, the protruding tail of T* may be available for reassociation with its complementary strand (T′), a process that may destabilise the probe-target duplex (T*P) as shown when asymmetrical products were used. This hybridisation behavior may also be observed with 3′ immobilised probes, although probes anchored to a support by a 3′ end are not commonly used.

Displacement of T* from P by reassociation with T′ may proceed through a sequential displacement pathway also known as a zipper effect (Reynaldo et al., 2000, J. Mol. Biol., 297:511-520). Hybridisation between the captured T* strand and its complementary strand T′ in solution would occur first at the exposed overhang tail of captured T* and would be followed by a branch migration mechanism. Such a mechanism was used recently to build a DNA-fueled nanomolecular machine (Yurke et al., 2000, Nature, 406:605-608; Alberti and Mergny, 2003, Proc. Nat. Acad. Sci. USA, 100:1569-1573). In those studies, the authors used the complementary DNA strand (called fuel DNA) to close and open double-stranded DNA structures. The complementary strand T′ may act as the fuel DNA, thereby pulling the captured target strand T* from the probe (FIG. 4).

By using asymmetrical PCR, we have shown that the captured product strand is displaced by the target complementary strand T′ independently of the area the probe targets on the product (FIG. 5). This suggests that some elements stabilise T*P when the hybridisations were performed in the presence of both T* and T′. One possible model would be that T*free forms a quaternary complex (T*T′T*free P) with the ternary complex (T′T*P) captured on the glass surface. In accordance with the random walk theory for branch migration (Lee et al., 1970, J. Mol. Biol. 48:1-22), the branch point between T*T′ and T′T*free duplexes of the T*T′T*free P complex may move in either direction. The random walk would continue until one of two helices becomes shorter than the minimum length of a stable duplex (Reynaldo et al., 2000, J. Mol. Biol., 297:511-520). This means that the longer the duplex part of the helix is, the more likely it is to displace the other competing duplex (e.g. if T*T′ forms a longer helix, it would destabilise the complex T′T free and vice-versa).

A nucleation step would occur first with encounter between T′ and the overhanging part of the captured T*. A double helix would rapidly be formed until it reaches the branch point made by the complex T*P (Radding et al., 1977, J. Mol. Biol., 116: 825-839). At that point, it is proposed that strand displacement by branch migration would start with the two complexes T*P and T′T*. Simultaneously, the T*free would form a double-stranded helix with the overhanging part of T′ associated with T*P (FIGS. 4 C and D), thereby forming an antagonist migration fork. When the 5′ overhang of T* DNA is longer (FIG. 4C), the double helix formed with T′ will be longer than the double helix formed between T*free and the overhanging part of T′. Branch mechanism competition between the two duplexes would be in favour of the reassociation of captured T* with T′, pulling the target T* away from the probe P. In contrast, when the 5′ overhanging tail is short (FIG. 4D), the competing forming helix T*freeT′ would be long enough to favour reassociation of T*free with T′, thereby depleting locally the T′ and thus stabilising the T*P complex. Over time, diffusion of T* in close proximity with free probes P, would feed the hybridisation of the target T* with the captured probe P, increasing the fluorescent signal (FIG. 3 A, B, C). The results presented herein provide evidence that kinetic effects involving re-association of the complementary nucleic acid strand may be associated with destabilisation of the capture probe/nucleic acid target duplex and that this kinetic effect may be governed by the position of the complementary sequence on the targeted nucleic acids. The results presented herein therefore delineate key predictable parameters that govern the hybridisation efficiency of capture probes attached onto solid supports. These parameters allow selection of optimal capture probes for the detection of nucleotide polymorphisms. The kinetic effects and reassociation of the target to the PCR product's complementary strand may lead to destabilisation of the capture probe/DNA target duplex (complex) and that this kinetic effect may be governed by the position of the complementary sequence on the targeted nucleic acid.

A correlation between the length of the overhang of the target and the efficiency of hybridization has been demonstrated herein. Evidence that the presence of the complementary strand is associated with the poor hybridisation efficiency of 5′ immobilised probes targeting the 3′ end of a product, thereby leaving a long 5′ overhang has also been evidenced herein. On the other hand, probes targeting a region of the target which is located toward the 5′ end of the same product, hybridised more efficiently. Therefore, the hybridisation efficiency of oligonucleotides anchored onto a solid support has been found to be highly dependent of their location on a target single-stranded nucleic acid. The results presented herein show that capture probes anchored by their 5′ end and targeting a region that lies within about a 40% portion of the 5′ end of the captured nucleic acid strand provide more efficient hybridisations as compared to those targeting the remaining 60% portion at the 3′ end (FIGS. 2, 6 and 7). Conversely, capture probes anchored by their 3′ end and targeting a region that lies within a 40% portion of the 3′ end of the captured nucleic acid strand provide more efficient hybridisations as compared to those targeting the remaining 60% portion at the 5′ end. Evaluation of the hybridisation signal for each probe revealed an inverse correlation between the length of the free overhanging end (either the 5′ end or the 3′ end depending on which end of the probe is anchored on the support) of the target and the hybridisation signal intensity. Therefore, hybridised targets having their longest portion (e.g., at least 60%) proximal the solid support have been found to be more stable and to give a better (more intense) hydridisation signal.

Results presented herein teach methods for the efficient design of capture probes, which help to improve the sensitivity and specificity of microarray detection. Methods used in the selection and design of probes, thus ensure efficient and sensitive detection of either target single-stranded nucleic acids or denatured double-stranded nucleic acids such as PCR amplicons. This study demonstrates the importance of choosing the appropriate nucleic acid region to ensure efficient and sensitive detection of a target such as single-stranded nucleotide-based target which may come into contact with a nucleotide sequence substantially complementary to the unhybridized portion of the target (which extends away form the support), or double-stranded DNA fragments such as PCR products using short capture probes. This is particularly important for SNP detection. In addition, efforts are ongoing to develop novel amplification and labeling systems for efficient production of single-stranded DNA products that would circumvent the competition between complementary strands.

Although the present invention has been described hereinabove by way of embodiments thereof, it may be modified, without departing from the spirit and nature of the subject invention as defined in the appended claims.

TABLE 1
Oligonucleotide primers and probes used in this invention.

| | | | Target | |
|---|---|---|---|---|
| Primers | SEQ ID NOs | Nucleotide sequence (5′ −−> 3′)^{b} | gene | Product length |
| ErmB225 | 1 | TCGTGTCACTTTAATTCACCAAGATA | ermB | 402 bp |
| ErmB601 | 2 | TTTTTAGTAAACAGTTGACGATATTC | ermB | (SEQ ID NOs 1 + 2) |
| ErmB109 | 3 | GGAACAGGTAAAGGGCATTTAACGAC | ermB | 433 bp |
| ErmB512 | 4 | CTGTGGTATGGCGGGTAAGTTTTATTAAG | ermB | (SEQ ID NOs 3 + 4) |
| TShoH240 | 5 | GCTTTAGAAGGCGATGCTCAATACG | tuf | 523 bp |
| TStaG765 | 6 | TIACCATTTCAGTACCTTCTGGTAA | tuf | (SEQ ID NOs 5 + 6) |
| shv604 | 7 | CAGCTGCTGCAGTGGATGGT | bla_{SHV} | 182 bp |
| | | | | (SEQ ID NOs 7 + 12) |
| shv449 | 8 | AGATCGGCGACAACGTCACC | bla_{SHV} | 337 bp |
| | | | | (SEQ ID NOs 8 + 12) |
| shv368 | 9 | TTACCATGAGCGATAACAGC | bla_{SHV} | 418 bp |
| | | | | (SEQ ID NOs 9 + 12) |
| shv313 | 10 | AGCGAAAAACACCTTGCCGAC | bla_{SHV} | 473 bp |
| | | | | (SEQ ID NOs 10 + 12) |
| shvseq71 | 11 | AGCCGCTTGAGCAAATTAAACTA | bla_{SHV} | 715 bp |
| | | | | (SEQ ID NOs 11 + 12) |
| shv763 | 12 | GTATCCCGCAGATAAATCACCAC | bla_{SHV} | |

Capture probes^{a}

| | | | |
|---|---|---|---|
| A-S-ErmBH272 | 13 | CAAACAGAGGTATAAAATTG | ermB |
| A-S-ErmBH370 | 14 | TGATTGTTGAAGAAGGATTC | ermB |
| A-S-ErmBH459 | 15 | TTGCTTAAGCTGCCAGCGGA | ermB |
| A-S-ErmBH272a | 16 | CAATTTTATACCTCTGTTTG | ermB |
| A-S-ErmBH370a | 17 | GAATCCTTCTTCAACAATCA | ermB |
| A-S-ErmBH459a | 18 | TCCGCTGGCAGCTTAAGCAA | ermB |
| A-S-TShoH713 | 19 | ATACGTTTTATCAAAAGATGAAG | tuf |
| A-S-TStaGH554 | 20 | TACTGGTGTAGAAATGTTC | tuf |
| A-S-TShoH520a | 21 | GAAGTTTCTTTGATACCAAT | tuf |
| A-S-TShoH520 | 22 | ATTGGTATCAAAGAAACTTC | tuf |
| A-S-shv1H691 | 23 | CCCCGCTCGCCAGCTCCGGT | bla_{SHV} |

^{a}A-S stands for the 5′ modifications: A is an amino group and S is a hexa-ethyleneglycol spacer.
^{b}Nucleotide nomenclature is as follows: A: Adenine; C: Cytosine; G: Guanine; I: Inosine; T: Thymine

### DESCRIPTION OF SEQUENCES

### 2) INFORMATION FOR SEQ ID NO: 1

(i) SEQUENCE CHARACTERISTICS:
(A) LENGTH: 26 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 1
TCGTGTCACT TTAATTCACC AAGATA

### 2) INFORMATION FOR SEQ ID NO: 2

(i) SEQUENCE CHARACTERISTICS:
(A) LENGTH: 26 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 2
TTTTTAGTAA ACAGTTGACG ATATTC

### 2) INFORMATION FOR SEQ ID NO: 3

(i) SEQUENCE CHARACTERISTICS:
(A) LENGTH: 26 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 3
GGAACAGGTA AAGGGCATTT AACGAC

### 2) INFORMATION FOR SEQ ID NO: 4

(i) SEQUENCE CHARACTERISTICS:
(A) LENGTH: 29 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 4
CTGTGGTATG GCGGGTAAGT TTTATTAAG

### 2) INFORMATION FOR SEQ ID NO: 5

(i) SEQUENCE CHARACTERISTICS:
(A) LENGTH: 25 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 5
GCTTTAGAAG GCGATGCTCA ATACG

### 2) INFORMATION FOR SEQ ID NO: 6

(i) SEQUENCE CHARACTERISTICS:
(A) LENGTH: 25 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 6
TIACCATTTC AGTACCTTCT GGTAA

### 2) INFORMATION FOR SEQ ID NO: 7

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 7
CAGCTGCTGC AGTGGATGGT

### 2) INFORMATION FOR SEQ ID NO: 8

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 8
AGATCGGCGA CAACGTCACC

### 2) INFORMATION FOR SEQ ID NO: 9

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 9
TTACCATGAG CGATAACAGC

### 2) INFORMATION FOR SEQ ID NO: 10

(i) SEQUENCE CHARACTERISTICS:
(A) LENGTH: 21 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 10
AGCGAAAAAC ACCTTGCCGA C

### 2) INFORMATION FOR SEQ ID NO: 11

(i) SEQUENCE CHARACTERISTICS:
(A) LENGTH: 23 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 11
AGCCGCTTGA GCAAATTAAA CTA

### 2) INFORMATION FOR SEQ ID NO: 12

(i) SEQUENCE CHARACTERISTICS:
(A) LENGTH: 23 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 12
GTATCCCGCA GATAAATCAC CAC

### 2) INFORMATION FOR SEQ ID NO: 13

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 13
CAAACAGAGG TATAAAATTG

### 2) INFORMATION FOR SEQ ID NO: 14

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 14
TGATTGTTGA AGAAGGATTC

### 2) INFORMATION FOR SEQ ID NO: 15

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 15
TTGCTTAAGC TGCCAGCGGA

### 2) INFORMATION FOR SEQ ID NO: 16

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 16
CAATTTTATA CCTCTGTTTG

### 2) INFORMATION FOR SEQ ID NO: 17

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 17
GAATCCTTCT TCAACAATCA

### 2) INFORMATION FOR SEQ ID NO: 18

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 18
TCCGCTGGCA GCTTAAGCAA

### 2) INFORMATION FOR SEQ ID NO: 19

(i) SEQUENCE CHARACTERISTICS:
(A) LENGTH: 23 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 19
ATACGTTTTA TCAAAAGATG AAG

### 2) INFORMATION FOR SEQ ID NO:20

(i) SEQUENCE CHARACTERISTICS:
(A) LENGTH: 19 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 20
TACTGGTGTA GAAATGTTC

### 2) INFORMATION FOR SEQ ID NO: 21

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 21
GAAGTTTCTT TGATACCAAT

### 2) INFORMATION FOR SEQ ID NO: 22

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 22
ATTGGTATCA AAGAAACTTC

### 2) INFORMATION FOR SEQ ID NO: 23

(i) SEQUENCE CHARACTERISTICS:
(A) LENGTH: 20 bases
(B) TYPE: Nucleic acid
(C) STRANDEDNESS: Single
(D) TOPOLOGY: Linear

(ii) MOLECULE TYPE: DNA

(xi) SEQUENCE DESCRIPTION: SEQ ID NO: 23
CCCCGCTCGC CAGCTCCGGT

1.-115. (canceled)

116. A method for increasing the efficiency of detection of at least one nucleic acid-based target, the method comprising;
a) providing a probe which is substantially complementary to a portion of a region located between nucleotide no. 1 and nucleotide no. n or between nucleotide no. m and nucleotide no. q of said target,
wherein n is defined according to the formula n=0.4q,
wherein m is defined according to the formula m=0.6q,
wherein q is the total nucleotide number of said target,
wherein when said capture probe is binding a region located between nucleotide no. 1 and nucleotide no. n of said target, said capture probe is anchored to the solid support by a probe's 5′ end thereof,
wherein when said capture probe is binding a region located between nucleotide no. m and nucleotide no. q of said target, said capture probe is anchored to the solid support by a probe's 3′ end thereof,
b) contacting said target and the solid support-anchored oligonucleotide-based capture probe,
wherein said capture probe generates a higher signal in comparison to a signal measured for a second capture probe which binds to a region outside of the region located between nucleotide no. 1 and nucleotide no. n or between nucleotide no. m and nucleotide no. q of said target and
wherein a signal intensity measured for a target hybridized to said capture probe is higher than a signal intensity measured for a substantially similar target hybridized to a second probe located outside of said region.

117. The method of claim 116, wherein said target comprises a label.

118. The method of claim 117, wherein said label generates a fluorescent signal.

119. The method of claim 116, further comprising detecting a complex formed by a hybridized capture probe and target.

120. The method of claim 116, wherein said target comprises an unhybridized portion of less than 1000 nucleotides.

121. The method of claim 116, wherein said contacting is carried out for more than 30 minutes.

122. The method of claim 116, wherein said capture probe has a AG of between 0 and −10 kcal/mol.

123. The method of claim 116, comprising increasing the efficiency of detection of a first nucleic acid-based target and a second nucleic acid-based target.

124. The method of claim 123, wherein a signal obtained for a first complex formed by a capture probe hybridized with a first nucleic acid-based target is compared with a signal obtained for a second complex formed by said capture probe hybridized with a second a nucleic acid-based target.

125. The method of claim 116, wherein said target comprises DNA, RNA, or a nucleic acid analog.

126. The method of claim 116, wherein said capture probe comprises DNA, RNA, or a nucleic acid analog.

127. The method of claim 116, wherein said target comprises deoxyribonucleotides, ribonucleotides, modified deoxyribonucleotides or modified ribonucleotides.

128. The method of claim 116, wherein said capture probe comprises deoxyribonucleotides, ribonucleotides, modified deoxyribonucleotides or modified ribonucleotides.

129. The method of claim 116 wherein said solid support is made from a material that is able to bind nucleic acids or analogs.

130. The method of claim 116, wherein said solid support is selected from the group consisting of glass, plastic, silicon, gold particles, beads and membranes.

131. The method of claim 116, wherein said target is a single-stranded nucleic acid.

132. The method of claim 116, wherein said target is a denatured double-stranded nucleic acid.

133. The method of claim 116, wherein said target is a PCR amplicon.

134. The method of claim 116, wherein said target is genomic DNA, cDNA, or RNA.

135. The method of claim 116, wherein said target nucleic acid is generated with a primer pair selected from the group consisting of a primer pair comprising SEQ ID NO.: 1, SEQ ID NO.: 2, SEQ ID NO.: 3 and SEQ ID NO.: 4, wherein said primer pair comprises at least one primer able to bind a sense strand of said target and one primer able to bind an anti-sense strand of said target.

136. The method of claim 116, wherein said capture probe comprises a sequence selected from the group consisting of SEQ ID NO.:14, SEQ ID NO.:15, SEQ ID NO.:16, SEQ ID NO.:17 and analogs thereof.

137. The method of claim 116, wherein said capture probe comprises a sequence selected from the group consisting of SEQ ID NO.:13, SEQ ID NO.:14, SEQ ID NO.:15, SEQ ID NO.:16, SEQ ID NO.:17, SEQ ID NO.:18 and analogs thereof and wherein said target is selected so that the probe binds a region located between nucleotide no. 1 and nucleotide no. n or between nucleotide no. m and nucleotide no. q of said target.

138. The method of claim 116, wherein said target nucleic acid is generated with a primer pair selected from the group consisting of a primer pair comprising SEQ ID NO.: 5, SEQ ID NO.: 6 and analogs thereof.

139. The method of claim 116, wherein said capture probe comprises SEQ ID NO.:19 or an analog thereof.

140. The method of claim 116, wherein said capture probe comprises a sequence selected from the group consisting of SEQ ID NO.:19, SEQ ID NO.:20, SEQ ID NO.:21, SEQ ID NO.:22 and analogs thereof and wherein said target is selected so that the probe binds a region located between nucleotide no. 1 and nucleotide no. n or between nucleotide no. m and nucleotide no. q of said target.

141. The method of claim 116, wherein said target nucleic acid is generated with a primer pair selected from the group consisting of a primer pair comprising SEQ ID NO.: 7, SEQ ID NO.: 8, SEQ ID NO.: 9, SEQ ID NO.: 10, SEQ ID NO.: 11, SEQ ID NO.: 12, and analogs thereof, wherein said primer pair comprises at least one primer able to bind a sense strand of said target and one primer able to bind an anti-sense strand of said target.

142. The method of claim 116, wherein said capture probe comprises a sequence selected from the group consisting of SEQ ID NO.:23 and analogs thereof.

143. The method of claim 116, wherein the closer said region is to nucleotide no. 1 or nucleotide no. q of said target, the higher the signal obtained.

144. A single-stranded oligonucleotide-based capture probe for detection of a target selected from the group consisting of a PCR amplicon of 550 nucleotides long or less from a ermB gene of a Staphylococcus aureus, a PCR amplicon of 600 nucleotides long or less from a tuf gene of a Staphylococcus species and a PCR amplicon of 1000 nucleotides long or less from a bla_{SHV} gene of a Escherichia coli., said capture probe able to bind to a substantially complementary target nucleotide sequence, whereby upon hybridisation of said capture probe and said target, a length of an unhybridized portion of said target which extends away from a solid support to which said capture probe is to be anchored, is 40% or less of the total length of said target.

145. The capture probe according to claim 144, wherein said capture probe is designed to bind a region located between nucleotide no. 1 and nucleotide no. n or between nucleotide no. m and nucleotide no. q of said target,
wherein n is defined according to the formula n=0.4q,
wherein m is defined according to the formula m=0.6q,
wherein q is the total nucleotide number of said target.

146. An array comprising the capture probe of claim 116.

147. A kit comprising the capture probe of claim 116.


SEQUENCE LISTING (23 sequences)

SEQ ID NO: 1 - DNA, 26 bp, ARTIFICIAL
  PCR PRIMER
tcgtgtcactttaattcaccaagata

SEQ ID NO: 2 - DNA, 26 bp, ARTIFICIAL
  PCR PRIMER
tttttagtaaacagttgacgatattc

SEQ ID NO: 3 - DNA, 26 bp, ARTIFICIAL
  PCR PRIMER
ggaacaggtaaagggcatttaacgac

SEQ ID NO: 4 - DNA, 29 bp, ARTIFICIAL
  PCR PRIMER
ctgtggtatggcgggtaagttttattaag

SEQ ID NO: 5 - DNA, 25 bp, ARTIFICIAL
  PCR PRIMER
gctttagaaggcgatgctcaatacg

SEQ ID NO: 6 - DNA, 25 bp, ARTIFICIAL
  PCR PRIMER
tnaccatttcagtaccttctggtaa

SEQ ID NO: 7 - DNA, 20 bp, ARTIFICIAL
  PCR PRIMER
cagctgctgcagtggatggt

SEQ ID NO: 8 - DNA, 20 bp, ARTIFICIAL
  PCR PRIMER
agatcggcgacaacgtcacc

SEQ ID NO: 9 - DNA, 20 bp, ARTIFICIAL
  PCR PRIMER
ttaccatgagcgataacagc

SEQ ID NO: 10 - DNA, 21 bp, ARTIFICIAL
  PCR PRIMER
agcgaaaaacaccttgccgac

SEQ ID NO: 11 - DNA, 23 bp, ARTIFICIAL
  PCR PRIMER
agccgcttgagcaaattaaacta

SEQ ID NO: 12 - DNA, 23 bp, ARTIFICIAL
  PCR PRIMER
gtatcccgcagataaatcaccac

SEQ ID NO: 13 - DNA, 20 bp, ARTIFICIAL
  CAPTURE PROBE
caaacagaggtataaaattg

SEQ ID NO: 14 - DNA, 20 bp, ARTIFICIAL
  CAPTURE PROBE
tgattgttgaagaaggattc

SEQ ID NO: 15 - DNA, 20 bp, ARTIFICIAL
  CAPTURE PROBE
ttgcttaagctgccagcgga

SEQ ID NO: 16 - DNA, 20 bp, ARTIFICIAL
  CAPTURE PROBE
caattttatacctctgtttg

SEQ ID NO: 17 - DNA, 20 bp, ARTIFICIAL
  CAPTURE PROBE
gaatccttcttcaacaatca

SEQ ID NO: 18 - DNA, 20 bp, ARTIFICIAL
  CAPTURE PROBE
tccgctggcagcttaagcaa

SEQ ID NO: 19 - DNA, 23 bp, ARTIFICIAL
  CAPTURE PROBE
atacgttttatcaaaagatgaag

SEQ ID NO: 20 - DNA, 19 bp, ARTIFICIAL
  CAPTURE PROBE
tactggtgtagaaatgttc

SEQ ID NO: 21 - DNA, 20 bp, ARTIFICIAL
  CAPTURE PROBE
gaagtttctttgataccaat

SEQ ID NO: 22 - DNA, 20 bp, ARTIFICIAL
  CAPTURE PROBE
attggtatcaaagaaacttc

SEQ ID NO: 23 - DNA, 20 bp, ARTIFICIAL
  CAPTURE PROBE
ccccgctcgccagctccggt